(19) United States
(12) Patent Application Publication
(10) Pub. No.: US 2007/0254007 A1
(43) Pub. Date: Nov. 1, 2007

(54) CHITOSAN/NANOCRYSTALLINE HYDROXYAPATITE COMPOSITE MICROSPHERE-BASED SCAFFOLDS

(76) Inventors: Joel D. Bumgardner, Memphis, TN (US); Betsy M. Chesnutt, Memphis, TN (US); Warren O. Haggard, Bartlett, TN (US); Youling Yuan, Memphis, TN (US); Tangadhar Moreshwar Utturkar, Memphis, TN (US); Benjamin Reves, Memphis, TN (US)

Correspondence Address: W. EDWARD RAMAGE, COMMERCE CENTER SUITE 1000, 211 COMMERCE ST, NASHVILLE, TN, 37201, US

(21) Appl. No.: 11/740,042

(22) Filed: Apr. 25, 2007

Related U.S. Application Data
(60) Provisional application No. 60/794,688, filed on Apr. 25, 2006.

(51) Int. Cl. A61K 33/42

(52) U.S. Cl. 424/423; 424/603

(57) ABSTRACT

A composite chitosan/nano-hydroxyapatite microsphere-based material used for bone-grafting and delivery of therapeutic agents. The composite material may be produced using co-precipitation methods. The composite material may be used to form scaffolds with significantly greater surface area and surface roughness than scaffolds composed of only chitosan. Composite scaffolds exhibit less swelling and greater toughness and flexibility than scaffolds fabricated by other techniques. Composite scaffolds also exhibit greater osteoblast proliferation. Composite scaffolds also may contain therapeutic agents or medicaments, and may be lyophilized.

This application claims priority to U.S. Provisional Patent Application No. 60/794,688, filed Apr. 25, 2006, by Joel D. Bumgardner, et al., and is entitled in whole or in part to that filing date for priority. The complete disclosure, specification and drawings of Provisional Patent Application No. 60/794,688 are incorporated herein in their entireties by reference.

### TECHNICAL FIELD

The present invention relates to a material for bone grafting. More particularly, the present invention relates to a composite material for use as a scaffold for bone engineering and as a vehicle for delivery of medicaments to a graft or wound site.

### BACKGROUND OF THE INVENTION

More than two million people in the United States each year suffer bone diseases, defects, or traumatic injuries that require orthopedic implants and/or bone grafting materials. The current gold standard for bone grafting is an autograft because there is no risk of disease transmission or immunological rejection. However, autografts are severely limited in quantity and sometimes quality, and they lead to pain and risk of infection at the donor site. Allografts are also popular. However, with allografts the risk of disease transmission and immunological reactions is present. Several synthetic materials are also currently being used as replacements for bone autografts and allografts. Calcium compounds such as calcium sulfate and calcium phosphate are some of the most commonly used materials. These materials are osteoconductive, but their degradation rate is difficult to control, and they are very brittle. Polymers such as polylactic and polyglycolic acid (PLA/PGA) and their copolymers are also being investigated as bone graft substitutes. However, these materials have been shown to release acidic degradation products that increase inflammation at the implant site and impair healing.

Many of these materials have also been investigated as vehicles to deliver therapeutic agents such as growth factors, antibiotics, or anesthetics to graft or implant sites. However, these biological compounds do not bind well to many of these materials, and because the degradation rate is difficult to control, the growth factors or other compounds are often released too quickly or not at a biologically driven rate.

One approach is to use porous biodegradable scaffolds that degrade at a rate similar to the rate of tissue regeneration, are osteoconductive, and have an open, interconnected pore structure with pores large enough to allow bone ingrowth, and sufficient mechanical strength to support healing tissues. Hydroxyapatite (HA) has been widely used as an orthopedic implant coating for more than two decades because of its osteoconductivity, but its use for bone grafting applications is limited to low loading conditions due to its brittle nature. Chitosan, a natural polymer, also has been investigated as a scaffold material because it is non-toxic, can be formed into complex structures, promotes cell adhesion and migration, enhances wound healing, and is biodegradable at a rate dependent on controllable factors such as degree of deacetylation, molecular weight, and crystallinity. However, although chitosan is tough and flexible, it lacks sufficient strength to be used alone in load bearing applications.

Several studies have investigated the use of composite chitosan/HA scaffolds. In general, composites of chitosan and HA or other forms of calcium phosphate can support bone cell growth and differentiation, but many of these scaffolds are produced by lyophilization, a method known to result in small pores, poor interconnected porosity, and weak mechanical properties.

The microstructure of composite chitosan/calcium phosphate materials also has an effect on their properties. Many composite scaffolds are composed of chitosan mixed with powdered HA or other forms of calcium phosphate, and others are chitosan scaffolds coated with HA. Weak interfacial bonding between chitosan and powdered CaP particles can result in decreased mechanical strength, and poorly integrated CaP particles may be able to migrate out of the chitosan matrix and cause inflammation and tissue damage. Co-precipitation methods may result in composites with both uniform distribution and strong interfacial bonding of nano-HA crystals in the chitosan matrix, but the pore size of such scaffolds is too small for effective bone ingrowth, and the lyophilization fabrication suffers from the problems described above.

Accordingly, what is needed is a bone graft material that overcomes the problems associated with other bone graft materials, particularly with a controllable degradation rate, the ability to bind biological compounds well, appropriate pore sizes, good interconnected porosity, and mechanical properties sufficient to support bone during healing.

### SUMMARY OF THE INVENTION

This invention is directed to a scaffold composite material combining chitosan with crystalline calcium phosphate. In one exemplary embodiment, the chitosan-calcium phosphate composite may be used in the surgical restoration of bone tissue that occurs due to birth defects, trauma, disease, implant revision, or similar events. The composite also may be used clinically with or without cells and with or without growth factors, antibiotics or other active agents for the treatment of an existing disease such as infection or cancer and/or to provide a stimulus for bone growth. The composite may be used to form a biodegradable scaffold with an organization that mimics the organic/inorganic nature of bone.

In another exemplary embodiment, the scaffold comprises a composite chitosan/nano-HA microsphere-based scaffold created by co-precipitation. The resulting scaffold possesses sufficient mechanical properties and an interconnected porous structure with pore sizes large enough to facilitate cell/tissue ingrowth. The scaffold promotes osteoblast attachment and proliferation.

In yet another exemplary embodiment, the scaffold or microsphere components of scaffold may be lyophilized to modulate biodegradation, release of medicaments, and tissue ingrowth. The microspheres also may be coated to extend the release of therapeutic agents and other medicaments therefrom.

### BRIEF DESCRIPTION OF THE DRAWINGS

FIG. 1 shows the chemical structure of chitin and chitosan monomeric units.

FIG. 2 shows a method of preparing composite microsphere scaffolds in accordance with an exemplary embodiment of the present invention.

FIG. 3 shows a flowchart of another method of preparing composite microsphere scaffolds in accordance with an exemplary embodiment of the present invention.

FIG. 4 shows scanning electron microscopy (SEM) images of chitosan and composite scaffolds in accordance with one exemplary embodiment of the present invention.

FIG. 5 shows representative micro-CT images of composite scaffolds in accordance with one exemplary embodiment of the present invention.

FIG. 6 shows representative EDS spectra of chitosan and composite scaffolds in accordance with one exemplary embodiment of the present invention.

FIG. 7 shows an SEM image of a composite scaffold with calcium and phosphorus elemental maps in accordance with one exemplary embodiment of the present invention.

FIG. 8 shows representative XRD spectra of chitosan and composite scaffolds in accordance with one exemplary embodiment of the present invention. Dashed arrows indicate peaks typical of chitosan, and solid arrows indicate peaks typical of hydroxyapatite. Indicated hydroxyapatite peaks are also labeled with their corresponding Miller indices.

FIG. 9 shows a representative TEM image of a composite scaffold in accordance with one exemplary embodiment of the present invention. Black areas are calcium phosphate crystals, and nano-calcium phosphate is distributed throughout the chitosan matrix.

FIG. 10 shows the average compressive modulus for hydrated (A) and dry (B) chitosan and composite scaffolds in accordance with one exemplary embodiment of the present invention. Asterisks (*) indicate statistical differences between groups (n=5, p<0.05).

FIG. 11 shows representative total calcium released and accumulated weight loss by chitosan and composite scaffolds in accordance with one exemplary embodiment of the present invention.

FIG. 12 shows fibronectin attachment to composite and chitosan scaffolds in accordance with one exemplary embodiment of the present invention. Asterisks (*) indicate statistical differences between groups (n=3, p<0.05).

FIGS. 13A-B show HEPM cell attachment (A) and proliferation (B) on chitosan and composite scaffolds in accordance with one exemplary embodiment of the present invention. Asterisks (*) indicate statistical differences between groups (n=4, p<0.05).

FIGS. 13C-D show Live/Dead staining of cells on composite (C) and chitosan (D) scaffolds in accordance with one exemplary embodiment of the present invention.

FIG. 14 shows ALP elution from scaffolds treated in various ways in accordance with various exemplary embodiments of the present invention.

FIG. 15 shows ALP elution from coated and uncoated scaffolds treated in various ways in accordance with various exemplary embodiments of the present invention.

FIG. 16 shows ALP elution from coated and uncoated scaffolds treated in various ways in accordance with various exemplary embodiments of the present invention.

FIG. 17 shows a representative SEM image of a lyophilized composite scaffold in accordance with one exemplary embodiment of the present invention.

FIG. 18 shows another representative SEM image of a lyophilized composite scaffold in accordance with one exemplary embodiment of the present invention.

FIG. 19 shows another representative SEM image of a lyophilized composite scaffold in accordance with one exemplary embodiment of the present invention.

### DETAILED DESCRIPTION OF EXEMPLARY EMBODIMENTS

In one exemplary embodiment, the invention described herein is a novel porous scaffold for use in bone tissue engineering that is composed of nanocrystalline CaP or hydroxyapatite (HA) in a chitosan matrix. Chitosan is a natural polymer that is biodegradable at a controlled rate dependent on its molecular weight and degree of deacetylation. It is non-toxic and biocompatible. It also has been shown to have some antibacterial, antifungal, and osteogenic properties, and both it and its degradation products enhance wound healing. In addition, chitosan can effectively accumulate and retain biologically active molecules and promote controlled release of those molecules due to its pH-dependent cationic nature.

Calcium phosphate is already used as a bone grafting material because it is osteoconductive, but alone, it is brittle. Composite chitosan and calcium phosphate microspheres possess the beneficial properties of both of these materials (e.g., biocompatibility, controllable degradation, and osteoconductivity, among others), that are important to bone tissue engineering/regeneration applications.

Calcium phosphate and chitosan have been combined previously by coating chitosan with a layer of calcium phosphate via precipitation from a saturated salt solution, or mixing powders of calcium phosphate into a chitosan solution prior to forming films, spheres, or fibers. The present invention is an improvement over these earlier technologies because the process allows for the formation of calcium phosphate at near nano-sized crystals that approximate the natural crystal structure in normal bone and that are well distributed throughout the chitosan matrix. This calcium-phosphate crystal size promotes cell proliferation and differentiation, and thus is advantageous to bone tissue engineering and regeneration applications. Also, because of the intimate connection between chitosan and calcium phosphate, improved mechanical properties of the composite are achieved. This is important since a bone tissue engineering scaffold must be able to support mechanical loading while new bone tissue is developing.

Another advantage of the present invention is that the composite material may formulated as microspheres that can be fused together to form complex shapes. This would allow custom grafts to be designed to fit any site.

In addition, therapeutic agents (e.g., growth factors, drugs, antibiotics, and other medicaments) may be added to the formation solutions to make composite microspheres containing said therapeutic agents. This allows those therapeutic agents to be released at a slower, more controlled rate, and to maintain a particular local concentration of the therapeutic agent for an extended period of time, as desired. Thus, for example, the invention may be used to maintain a high concentration of the therapeutic agent for a longer period of time than using current methods.

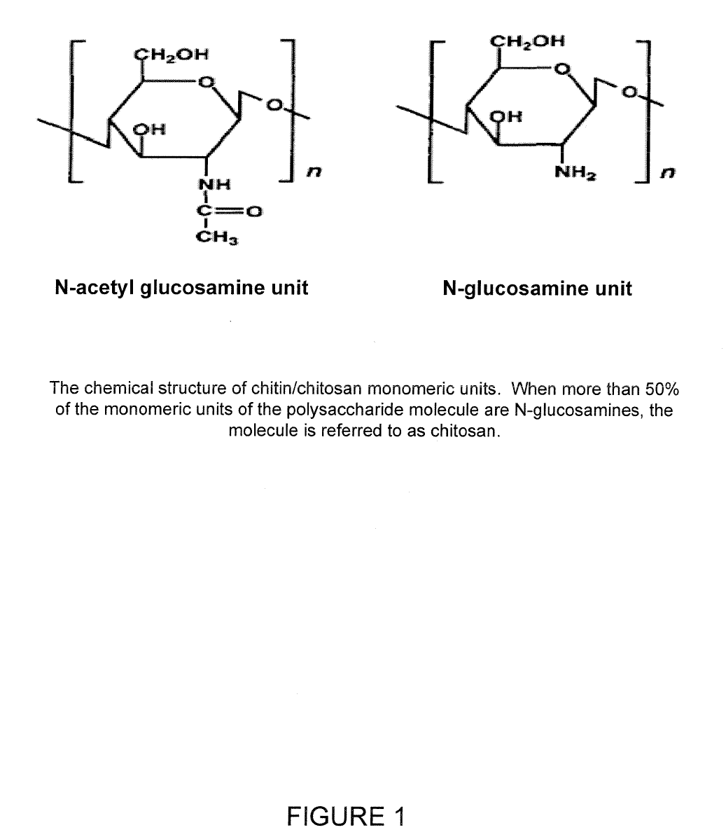

As seen in FIG. 1, chitosan is a linear polysaccharide co-polymer of N-acetyl-glucosamine and N-glucosamine units. Either an acetamido group (—NH—COCH₃) or an amino group (—NH₂) is attached to the C-2 carbon of the glucopyran ring. The degree of deacetylation (DDA) represents the percentage of amino groups attached to the polymer glucopyran rings. When more than 50% of the C-2 attachment is an amino group, i.e. >50% DDA, the material is termed chitosan. When more than 50% of the C-2 attachment is the acetomido group, i.e. >50% acetylated, the material is termed chitin.

The DDA is important to the physiochemical properties of the polysaccharide. Chitosan is soluble in aqueous solutions at pH<6, whereas chitin is not soluble in aqueous solutions. Crystallinity of the chitosan molecule increases with increasing DDA, resulting in polymers with higher strengths, lower moisture content and swelling properties, and lower susceptibility to degradation in physiological environments. Therefore, chitosan-calcium phosphate microspheres can be made to increase or decrease mechanical and swelling properties and degradation rates by selecting chitosans of different DDA to make the composite spheres to meet specific graft substitute and/or drug delivery applications.

The microspheres in accordance with one exemplary embodiment of the present invention can be used alone, as part of other delivery vessels (such as calcium sulfate or calcium phosphate), or formed into a scaffold of any size or shape to fit exactly into the graft site. The composite of chitosan with nano-sized calcium phosphate presents multiple development opportunities for musculoskeletal treatments. The resulting microspheres may be packed into many forms to make shapes as required for implant applications (e.g., the shape of a palate to treat cleft palate defects), or may be applied in the standard manner of other calcium compounds. The present invention thus has the ability to act both as a degradable bone graft and/or a drug delivery device that can be designed with flexible degradation and elution properties.

As discussed in greater detail below, testing of samples of various embodiments of scaffolds created in accordance with the present invention have shown an interconnected porous structure with pore sizes that can facilitate bone ingrowth. The resulting scaffolds are composed entirely of biocompatible, biodegradable materials, but does not degrade quickly even in the presence of lysozyme. Osteoblast cells are able to attach and grow well on the composite scaffold, and begin to grow into the interior pores quickly. Furthermore, as shown by dsDNA analysis, cell growth is significantly increased on the composite scaffolds.

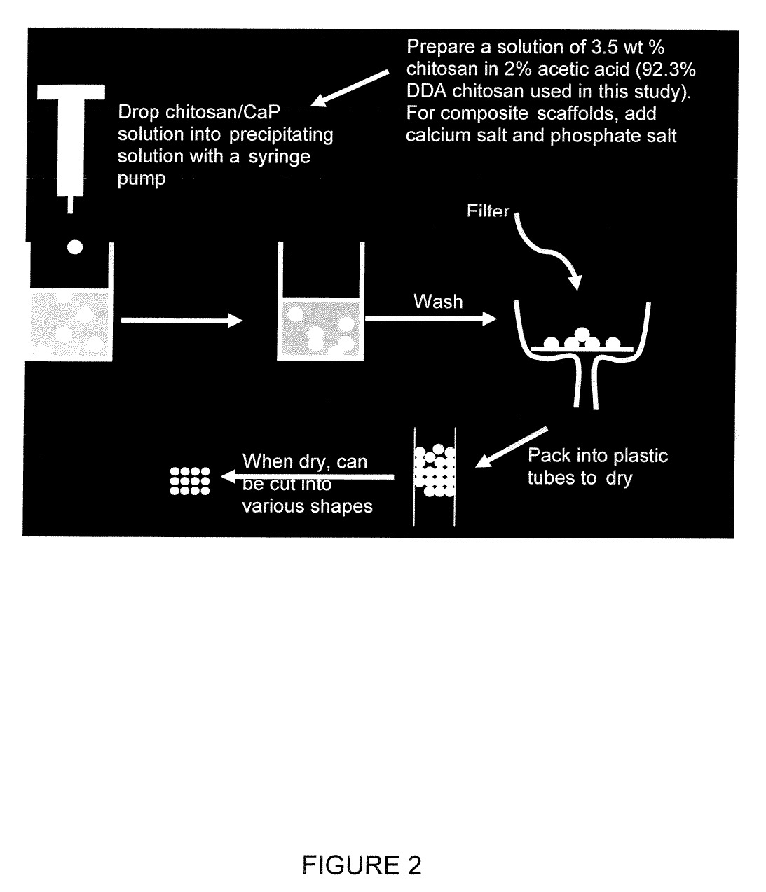

As shown in FIG. 2, the general method for forming chitosan-calcium phosphate composite microspheres involves preparing a chitosan/CaP solution with appropriate salts (which may include, but are not limited to, calcium salts and phosphate salts). The solution may be a 2% acetic acid solution as shown in the figure, but the concentration may vary, and the solution may be formed from other organic acid solvents as various concentrations. Similarly, while 92.3% DDA chitosan is shown in FIG. 2, chitosan with a different DDA may be used, and the weight % of chitosan in the solution may vary from 3.5 weight %.

The chitosan/CaP solution is then dropped into a precipitating solution using various means known in the art, such as one or more syringe pumps. This results in the formation of the composite microspheres, which are then left in the precipitating solution for an appropriate period of time to promote the development of crystalline CaP. The microspheres are removed and treated until a neutral pH is reached. This treatment may comprise washing with distilled de-ionized (DI) water until a neutral pH is reached. The microspheres are then quickly washed with an organic acid of relatively low concentration (such as, but not limited to, 1% acetic acid), in order to dissolve the surface of the microspheres slightly so they are able to stick together to form a porous scaffold of the desired shape or configuration, which may be accomplished by placing the microspheres in moulds, trays, or similar means of shape formation, and drying.

### EXAMPLE 1

Chitosan-calcium phosphate composite microspheres are made by dripping a 3.5 wt % chitosan (92.3% DDA) in 2% acetic acid solution containing 100 mM CaCl₂, and 60 mM NaH₂PO₄ into a NaOH/methanol solution. The chitosan-calcium phosphate microspheres are left in the NaOH/methanol solution for 24 hours to allow the initial amorphous CaP to develop into crystalline hydroxyapatite (HA). Then, the microspheres are washed with distilled de-ionized (DI) water until a neutral pH is reached. The microspheres are then quickly washed with 1% acetic acid, packed into 13 mm diameter tubes, and dried at room temperature.

### EXAMPLE 2

Composite chitosan/calcium phosphate scaffolds are formed as follows: 3.57 g of 92.3% DDA chitosan powder was dissolved in 84 mL 2 wt % acetic acid. 10 mL of 1M CaCl₂ in 2% acetic and 6 mL of IM NaH₂PO₄ in 2% acetic acid was added to the chitosan solution to give a final chitosan concentration of 3.44 wt %, a final Ca²⁺ concentration of 0.1 M, and a final PO₄⁻ concentration of 0.06 M (Ca:P ratio=1.67). The chitosan solution was placed in a 10 mL syringe fitted with a 21G needle (BD Medical, Franklin Lakes, N.J.). The syringe was placed in a syringe pump and the chitosan was slowly dripped into a solution composed of 20% NaOH, 30% methanol, and 50% water (pH=13), with constant stirring. This solution caused the chitosan drops to precipitate into solid beads. The beads were left in the basic solution for 24 hours to allow crystalline hydroxyapatite to develop from unstable brushite and amorphous calcium phosphate (ACP), likely according to the following reactions:
10CaHPO₄+12OH⁻→Ca₁₀(PO₄)₆(OH)₂+10H₂O+4PO₄³⁻
PO₄³⁻+ACP+OH⁻→Ca₁₀(PO₄)₆(OH)₂

After 24 hours, the beads were washed with DI water until they reached a neutral pH (7.0-7.5). They were then rinsed with 1 wt % acetic acid for 10 seconds and then filtered using a vacuum flask. The acetic acid wash is used to partially dissolve the surface of the beads and allow them to fuse together, forming a porous scaffold. For subsequent testing, the beads were packed into plastic tubes 13 mm in diameter that were open at both ends and dried at room temperature for at least three days to form cylindrical scaffolds. Scaffolds in other shapes can be fashioned, or the material may be applied in situ.

After the scaffolds were completely dry, they were rehydrated for four hours in deionized water and then cut into sections five mm thick. The scaffolds were then completely dried again and stored at room temperature.

Chitosan scaffolds containing no calcium phosphate were also prepared in a similar manner, with an initial solution that contained 3.57 g chitosan in 100 mL 2 wt % acetic acid.

The structure and surface morphology of the chitosan and composite scaffolds created as described above was examined by scanning electron microscopy (SEM). As shown in FIGS. 2A-2C, both types of scaffold were composed of fused particles 10 approximately 500 to 900 μm in diameter (FIG. 2A shows an SEM image of a chitosan scaffold at 20×, FIG. 2B shows a composite scaffold at 20×, and FIG. 2C shows the rough surface morphology of a composite scaffold at 2000×). These particles were approximately spherical. Pore sizes ranged from approximately 100 to 800 μm, and the overall porosities of both types of scaffold were similar (35.5±6.7% for chitosan scaffolds, and 33.7±5.2% for composite scaffolds).

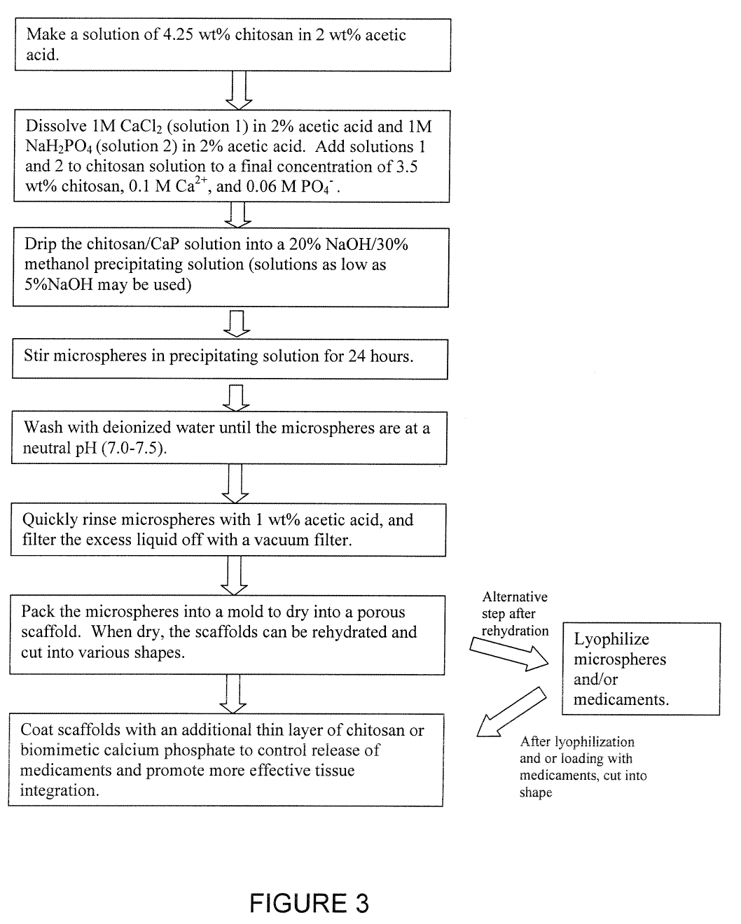

Micro-CT imaging demonstrated that chitosan and composite scaffolds had an internal interconnected porous structure. Representative slices in the axial (XY), coronal (XZ), and sagittal (YZ) planes are shown for a composite scaffold in FIG. 3.

Although the particle size and porosity were similar, the surface morphology of the scaffolds was very different. The chitosan scaffolds had a very smooth surface, while the surface of composite scaffolds was more textured, as shown in FIGS. 2A and 2B. This translated into significant differences in total surface area, with chitosan scaffolds having only 0.028±0.01 m²/g and composite scaffolds having more than twenty times this value with 0.707±0.16 m²/g (p<0.05).

The water content of both types of scaffold was similar, but the swelling ratio was significantly less for composite scaffolds (160.3±5.5%) than for chitosan scaffolds (176.0±7.9%) (p<0.05).

This data for this example is summarized in the following table:

| | | | Water | |
|---|---|---|---|---|
| Scaffold Type | Surface area (m2/g) | Porosity | Content | Swelling Ratio |
| Chitosan | 0.028 ± 0.01^{a} | 35.5 ± 6.7%^{c} | 17.2 ± 0.5%^{d} | 176.0 ± 7.9%^{e} |
| Composite | 0.707 ± 0.16^{b} | 33.7 ± 5.2%^{c} | 17.1 ± 0.2%^{d} | 160.3 ± 5.5%^{f} |

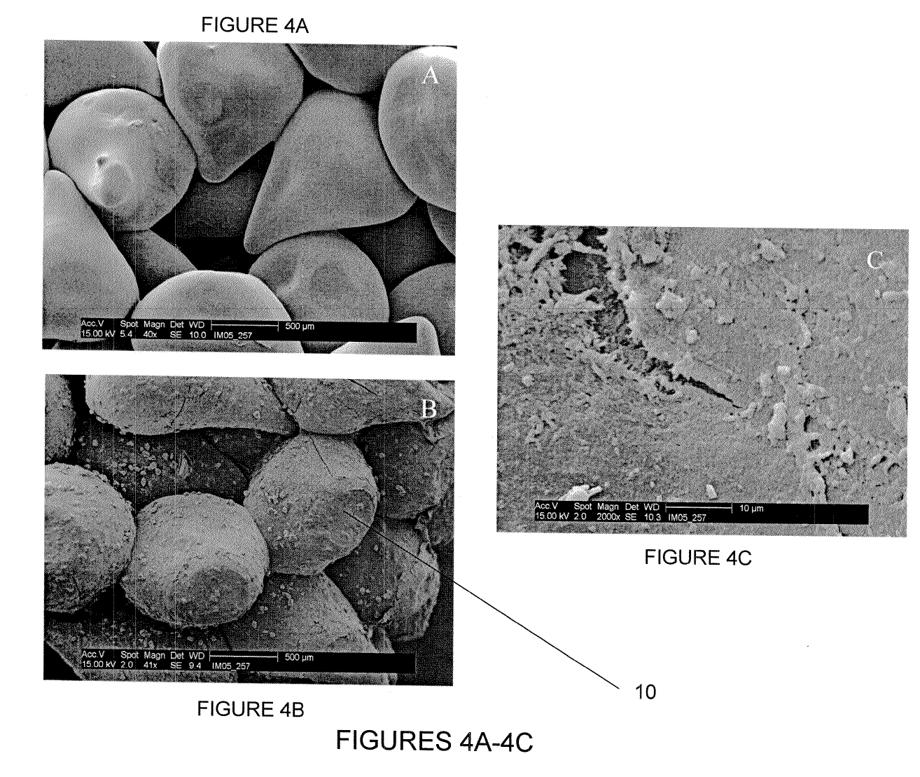

The Ca:P ratio of the scaffolds was determined by energy dispersive spectroscopy (EDS) to be 2.0±0.1. Representative spectra for chitosan (FIG. 4A) and composite (FIG. 4B) scaffolds are shown in FIG. 4. Scaffolds composed of only chitosan showed no calcium or phosphorus peaks while composite scaffolds had evident calcium and phosphorus peaks.

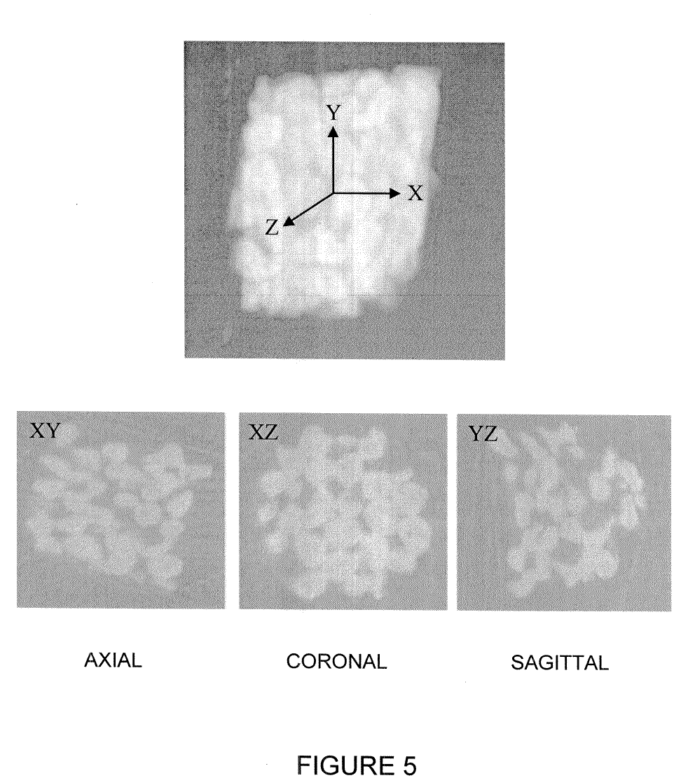

To examine the distribution of calcium and phosphorus in the scaffold, elemental mapping was used. As shown in FIGS. 5A-C, calcium and phosphorus were distributed throughout the scaffold. FIG. 5A shows a 2000× image of a cross-section of a composite scaffold. FIGS. 5B and 5C show elemental maps of calcium and phosphorus for the same area. From these images, no areas could be identified where calcium phosphate was not present.

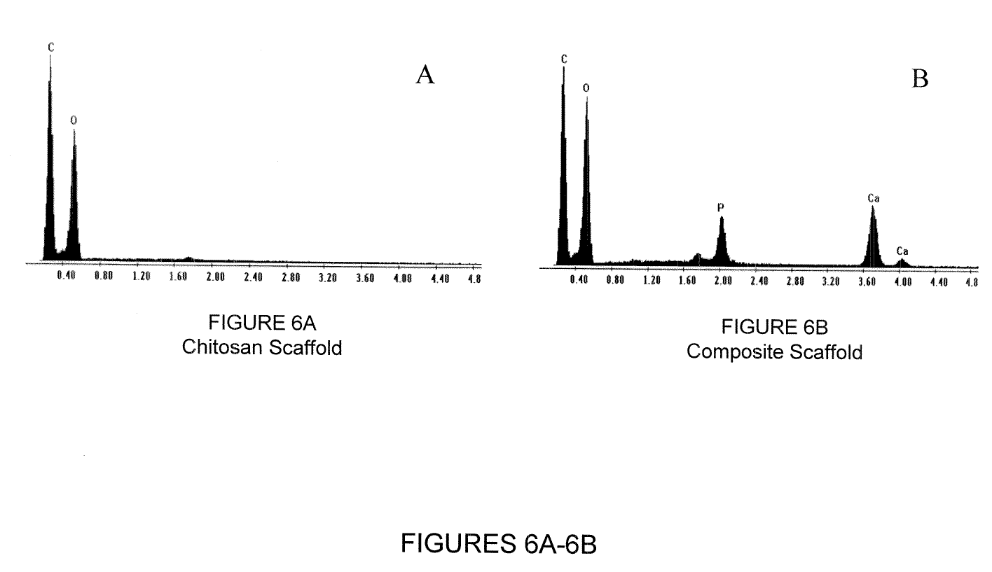

The crystallinity of the chitosan and calcium phosphate phases was determined by x-ray diffraction (XRD). FIG. 6A shows a representative spectrum for a chitosan scaffold, and FIG. 6B shows a representative spectrum for a composite scaffold. Peaks characteristic of chitosan are identified by dashed arrows and peaks characteristic of hydroxyapatite are identified by solid arrows. In the chitosan scaffold, peaks were seen at 2θ=20° and 2θ=10° (indicated by the dashed arrows in FIG. 6A). These peaks are typical of chitosan and the large peak at 2θ=20° was used to calculate the crystallinity index. The chitosan crystallinity index of scaffolds containing only chitosan was 79.7±1.2%, and this decreased to 67.3±1.5% for composite scaffolds. In composite scaffolds, peaks characteristic of hydroxyapatite could be identified (indicated by the solid arrows in FIG. 6B), and the crystallinity index of the calcium phosphate was estimated to be 16.7±6.8%. The average crystallite size was estimated to be 128±55 nm.

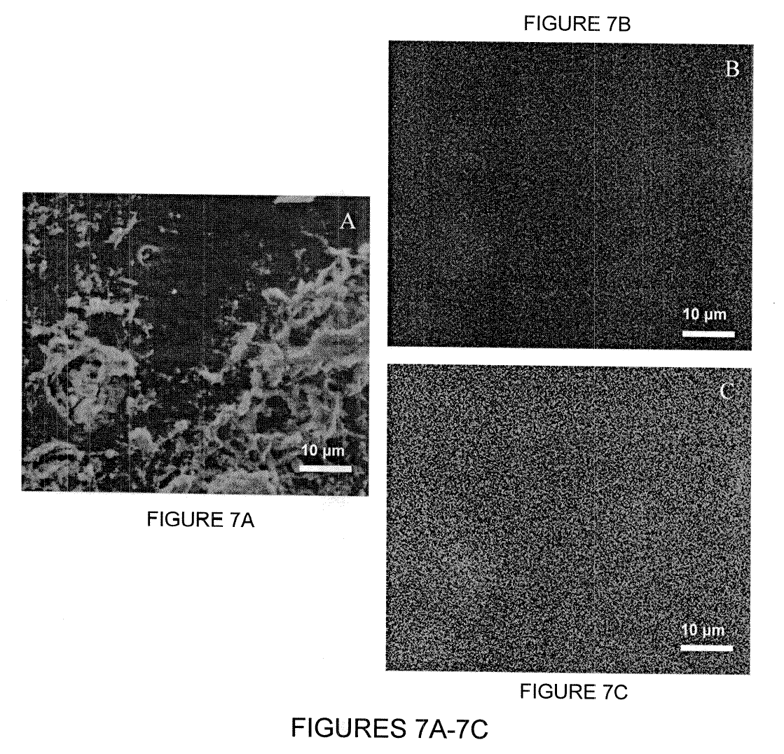

To confirm that calcium phosphate was nano-crystalline and distributed throughout the chitosan, cross-sections of microspheres were examined by transmission electron microscopy (TEM). FIG. 7 shows a representative TEM image of a composite microsphere. In this image, calcium phosphate crystals appear black, and are distributed throughout the chitosan matrix. Furthermore, crystals also appear to be approximately 100 nm or less in size.

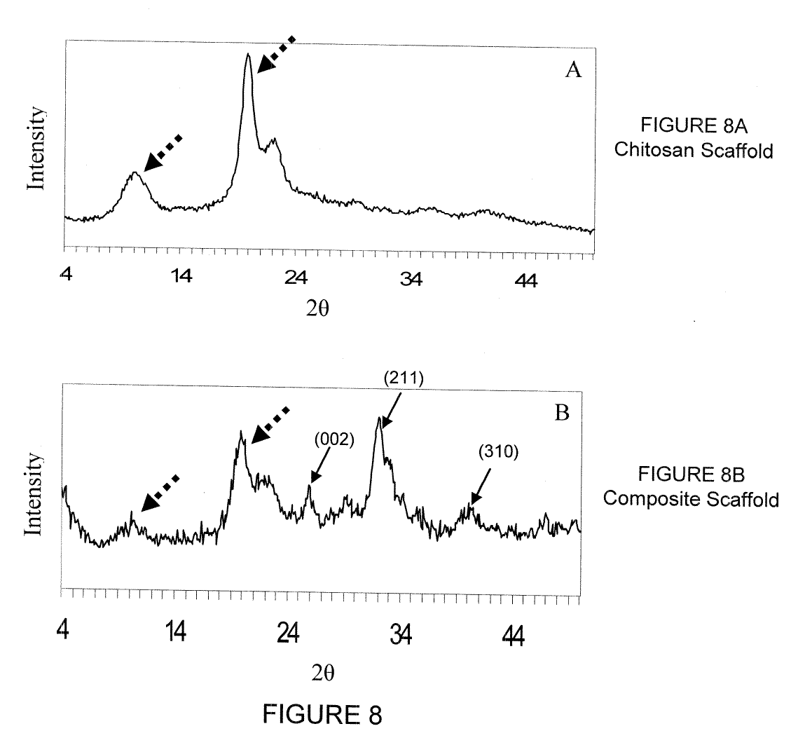

The compressive modulus of dry scaffolds and scaffolds rehydrated in phosphate buffered saline (PBS) was measured by compressing to 50% strain. The compressive modulus of rehydrated composite scaffolds was significantly higher than that of rehydrated chitosan scaffolds (9.29±0.8 MPa vs. 3.26±2.5 MPa) (as shown in FIG. 8A). The modulus of hydrated composite scaffolds approached the values that have been reported for cancellous bone (i.e., 10-2000 MPa). The hydrated scaffolds also were not brittle, and no scaffold broke during the compression testing. The compressive modulus of dry scaffolds was an order of magnitude higher than hydrated scaffolds for both chitosan (89.48±43.1 MPa) and composite scaffolds (117.57±52.8 MPa), and the dry scaffolds broke at 10-15% strain (as shown in FIG. 8B).

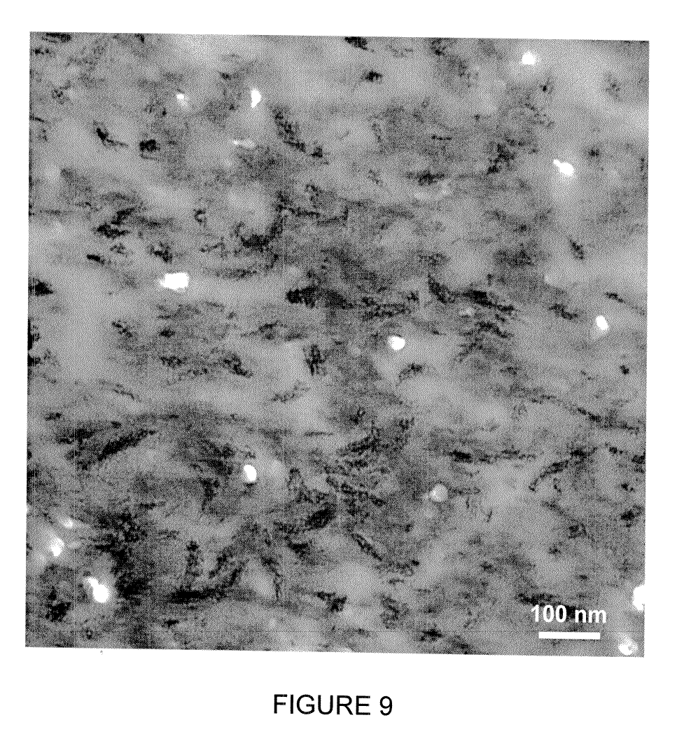

Degradation of scaffolds in PBS containing lysozyme was examined over 14 days. After one, four, seven, and fourteen days, scaffolds were removed and dried, and the mass was measured again to determine mass lost during degradation. There was no significant mass loss detected for either type of scaffold during the 14 day period (as shown in FIG. 9B). Calcium released into the media was also measured every other day (as shown in FIG. 9A). The total amount of calcium released from composite scaffolds was very low, less than 1% of the initial calcium present. Some calcium was also released from chitosan scaffolds at the first time point. SEM images showed that the morphology of the scaffolds had not appreciably changed. The Ca:P ratio (1.98) was very similar to the ratio before dissolution (2.01), and elemental mapping showed that calcium and phosphorus were still distributed evenly throughout the scaffold.

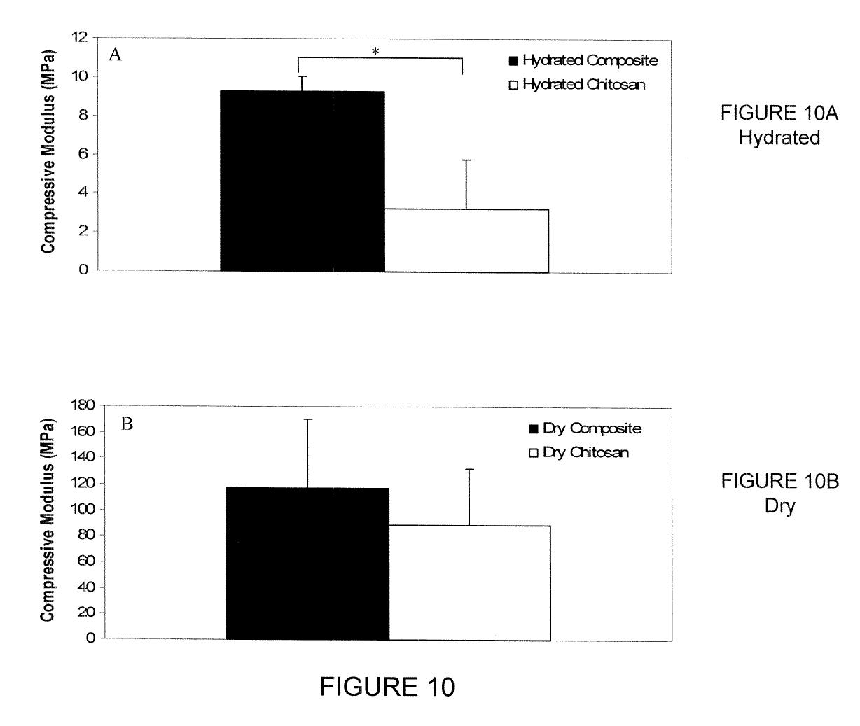

Adsorption of fibronectin, a protein known to be important for osteoblast adhesion, was examined over four hours. The total amount of adsorbed protein was measured after 30 minutes, one, two, and four hours. There were no differences in the amount of fibronectin adsorbed onto chitosan or composite scaffolds up to two hours (as shown in FIG. 10). However, after four hours, significantly more fibronectin had adsorbed onto composite scaffolds than chitosan scaffolds (p<0.05).

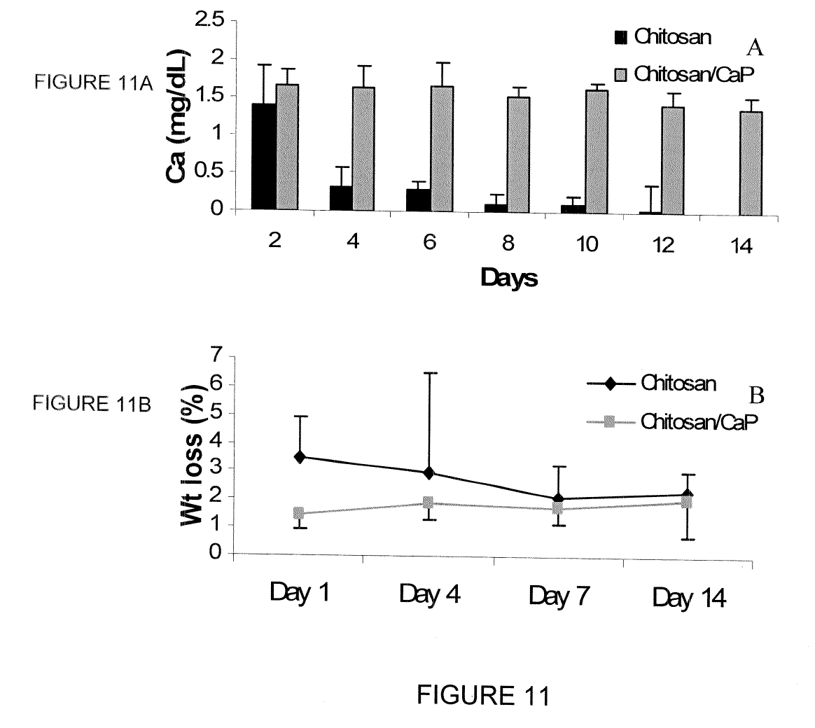

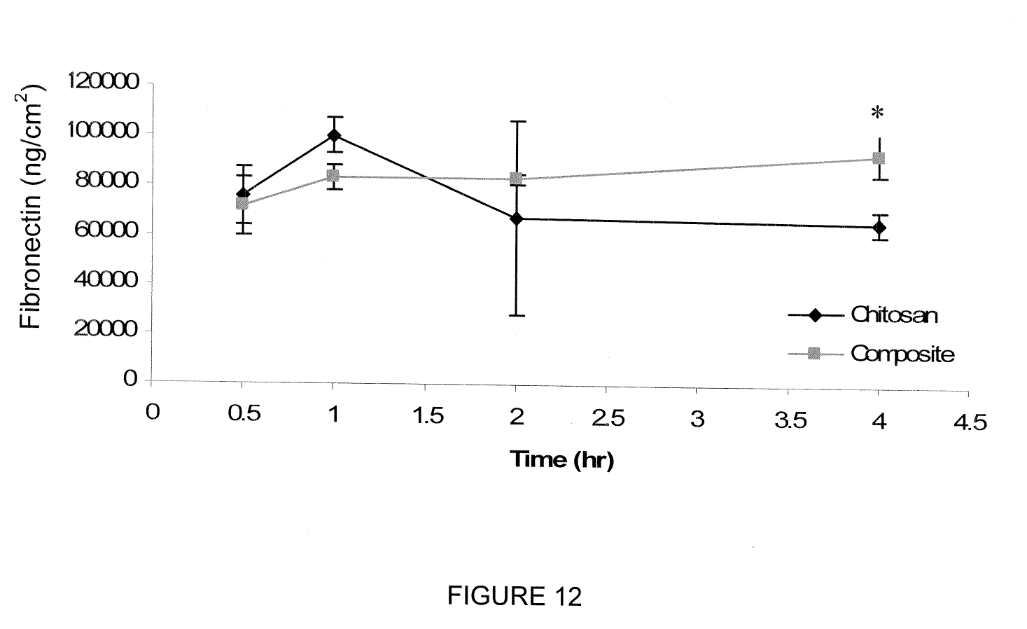

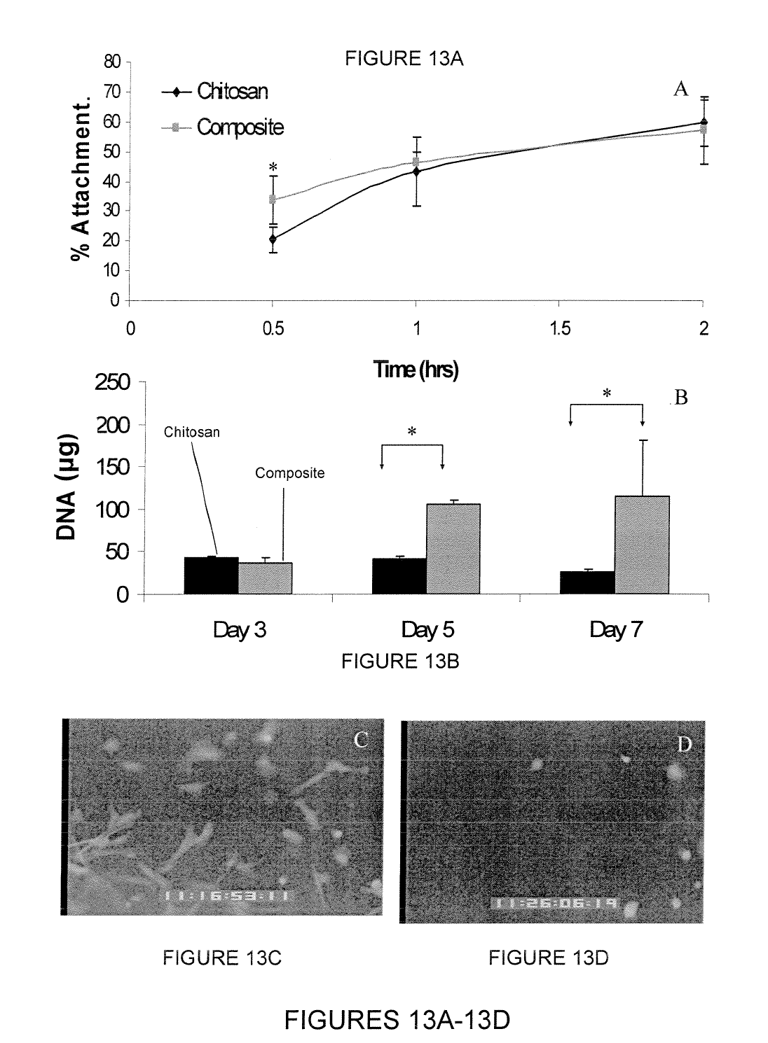

Cell attachment and proliferation on chitosan and composite scaffolds was examined with an osteoblastic-like cell line (ATCC CRL 1486 human embryonic palatal mesenchyme (HEPM) cell, Rockville, Md.). Cell attachment was measured after 0.5, 1, and 2 hours by counting the number of cells that were not attached. After 0.5 hours, the percentage of cells adhering to composite scaffolds was significantly higher than the number adhering to chitosan scaffolds (as shown in FIG. 11A). However, at one and two hours, the percent of attached cells was similar between the two scaffolds, and cells attached well to both types of scaffold with greater than 60% attachment after two hours. Cell proliferation was measured by measuring total double-stranded DNA (dsDNA) after three, five, and seven days. In contrast to the data for cell attachment, significantly more DNA was measured from cells cultured on composite scaffolds than on chitosan scaffolds after five and seven days (p<0.05) (as shown in FIG. 11B). This was confirmed by Live/Dead (InVitrogen Corp., Carlsbad, Calif.) staining in which live cells fluoresce green and dead cells fluoresce red. As shown in FIGS. 11C and 11D, many more live cells could be seen on composite scaffolds (FIG. 11C) than on chitosan scaffolds (FIG. 11D).

As demonstrated above, composite scaffolds with nano-scale calcium phosphate particles well integrated and distributed in a chitosan matrix can be produced using a co-precipitation method. As shown by elemental mapping, calcium and phosphorus were evenly distributed throughout these scaffolds in intimate association with the chitosan matrix. No areas were found where calcium phosphate was not present. Analysis by XRD demonstrated that the calcium phosphate phase in the composite was partially crystalline HA (16.7%). In some embodiments, the Ca:P ratio of composite scaffolds may be higher than the Ca:P ratio of pure hydroxyapatite (1.67), indicating that these scaffolds may contain some amorphous CaP as well as crystalline HA.

The calcium phosphate crystals developed were at the nano-scale and were well distributed throughout the matrix. It has been reported that inorganic crystallites precipitated in polymer matrices such as chitosan and gelatin exhibit strong chemical interactions via covalent bonding, ion-dipole interactions, and complexation of Ca²⁺ ions with polymer amino, acetylamino, and hydroxyl groups. The close association of calcium phosphate nanoparticles with chitosan and direct chemical bonding between organic and inorganic phases may limit the ability of the nanoparticles to migrate away from the scaffold implant and result in improved mechanical properties and decreased tissue damage.

The composite chitosan/nano-calcium phosphate microsphere-based scaffold described herein in accordance with one embodiment of the present invention is a significant improvement over earlier scaffolds because of its interconnected porosity and improved mechanical properties. This scaffold has an interconnected porous structure with pore sizes that are known to be able to support bone and vascular ingrowth (i.e., greater than approximately 100 μm). The compressive modulus of hydrated composite scaffolds created as described herein is higher than that of comparable chitosan scaffolds, and both composite and chitosan microsphere scaffolds created as describe herein exhibited higher compressive strengths than what has been reported for chitosan or composite chitosan/calcium phosphate scaffolds produced by lyophilization.

The modulus of dry composite scaffolds is an order of magnitude higher than the modulus of hydrated scaffolds and well within the range of cancellous bone. However, dry scaffolds are much more brittle and easily broken than hydrated scaffolds. In many prior art studies, the mechanical properties of polymeric scaffolds were only measured when the scaffolds were dry. However, after implantation polymeric materials will quickly hydrate and this has the potential to significantly change the mechanical properties. The composite scaffolds examined in this study showed a very significant drop in compressive modulus after rehydration. However, the presence of calcium phosphate reduced this effect, and composite scaffolds were still able to maintain values approaching those measured in human cancellous bone.

The mechanical properties of the composite scaffolds may be further modified by changing the size of the microspheres, the ratio of chitosan to calcium phosphate, or the pH at which nanocrystals are developed.

Microsphere-based composite scaffolds produced in accordance with one embodiment of the present invention were very tough, and no hydrated scaffold broke when compressed up to 50% of its original height, whereas scaffolds composed of only calcium phosphate are very brittle and easily broken. The use of chitosan polymer as the matrix provided the scaffolds with toughness and flexibility, while the addition of nano-calcium phosphate particles provided the necessary compressive modulus and strength. The increased flexibility and toughness make it unlikely that these scaffolds would break during healing, and therefore, they are more suitable than some stronger, more brittle materials.

The addition of HA into the microspheres did not change the size and shape of the particles and did not inhibit microsphere fusion. In fact, scaffolds composed of composite microspheres were much less likely to break during fabrication and processing than scaffolds composed of only chitosan microspheres. The presence of HA did, however, change the surface area and surface texture of the scaffolds. Composite scaffolds had more than twenty times more surface area than chitosan scaffolds. Osteoblast attachment and bone bonding are increased on rough surfaces, particularly surfaces that exhibit roughness on a nano-scale. The is seen by cell attachment being significantly increased on composite scaffolds at an early time point of thirty minutes, and by osteoblast proliferation similarly being significantly increased on composite scaffolds.

The swelling ratio of composite scaffolds created in accordance with an embodiment of the present invention also is significantly less than the swelling ratio of chitosan scaffolds. It is important for scaffolds to maintain their shape in vivo, and this has been reported to be a problem for chitosan-based scaffolds in the prior art. The decreased swelling rate of composite scaffolds indicates that they will be better able to maintain their shape after implantation than scaffolds composed of only chitosan. The swelling rate may be further controlled by altering the ratio of chitosan to calcium phosphate or cross-linking the microspheres.

The DDA and degradation rate of the chitosan used to form the composite scaffolds are important factors to be considered. The rate of degradation of composite scaffolds, and, consequently, the biological response to those scaffolds, may thus be influenced by the chitosan used. A low degradation rate may be achieved by using chitosan with a high DDA and crystallinity.

Lyophilized composite chitosan/calcium phosphate scaffolds may be formed by adding a lyophilization step to the above-described methods. This step comprises taking the resulting scaffolds, rehydrating them (if necessary), pre-freezing them (while the examples below demonstrate pre-freezing at −20° C. or −80° C., the actual pre-freezing temperature may be any other suitable temperature), and then freeze-drying them. In addition, the scaffold microspheres may subsequently be loaded with a therapeutic agent or other medicament, and coated (although coating is not necessary, and un-lyophilized scaffolds may be loaded and coated as well). The coating may be, but is not limited to, a thin layer of chitosan or biomimetic calcium phosphate. A specific example of this method is shown in FIG. 3. Additional examples showing the formation of testing samples at different temperatures, with and without coating, are described below.

### EXAMPLE 3

3.57 g of 92.3% degree of deacetylation chitosan is dissolved in 84 mL of 2 wt % acetic acid overnight. A 1 M sodium monobasic phosphate solution was made by dissolving 0.83 g in 6 mL 2 wt % acetic acid, and a 1 M calcium chloride solution was prepared by dissolving 1.47 in 10 mL 2 wt % acetic acid. The solutions are added to the chitosan solution and stirred. The resulting solution is dripped into a 20% NaOH/30% methanol/50% H₂O precipitating solution using a syringe pump or similar means. When the chitosan solution drips into basic solution, chitosan microspheres instantly precipitate. This process is repeated until all of the chitosan solution has been used. Once all of the microspheres have been made, they are left in the basic solution for 24 hours to allow crystalline hydroxyapatite to form. After 24 hours, the microspheres are washed numerous times with deionized water (DI water) to reduce the pH to neutral.

After fabrication, the microspheres are rinsed with 1 wt % acetic acid (or some other organic acid) for 10 seconds and then filtered using a vacuum flask. The acetic acid wash is used to partially dissolve the surface of the beads and allow them to fuse together, forming a porous scaffold. For subsequent testing, the beads were packed into centrifuge tubes that were open at both ends and dried at room temperature for approximately two to three days to form cylindrical scaffolds. Scaffolds also may be dried by other means, for different periods of time. Scaffolds in other shapes can be fashioned, or the material may be applied in situ.

In order to lyophilize, the scaffolds are rehydrated, such as in small glass vials. DI water is added until the vials are approximately two-thirds full. The vials are then either placed in a −20° C. or −80° C. freezer for pre-freezing. The pre-freezing step is allowed to continue for a few hours. The pre-frozen microspheres are then placed into the interior chamber of a freeze dryer. The freeze-drying step is allowed to continue for 48 hours. If freeze-drying is attempted before the scaffolds are allowed to air-dry first, lyophilization does not work.

Lyophilization drastically changes some of the properties of the microspheres. The density is lowered, porosity increased, and swelling ratio greatly increased. The freeze-dried composite microspheres have a very high medicament loading capacity. This loading capacity can be used for delivery of therapeutic agents such as BMP-2, other growth factors, drugs, or antibiotics.

The following table shows a comparison of the density and swelling ratio for lyophilized composite microspheres (pre-frozen at −20° C. and −80° C.) as compared to un-lyophilized composite microspheres, which were air dried after formation.

Density Determination and Swelling Ratio

| | Density (g/mL) | Swelling Ratio (%) |
|---|---|---|
| Air-dried | 2.29 ± 0.12 | 175 ± 7.0 |
| Pre-frozen at −20° C. | 0.40 ± 0.06 | 468 ± 13.9 |
| Pre-frozen at −80° C. | 0.37 ± 0.01 | 432 ± 2.62 |

The lyophilized microspheres have a greatly decreased density compared to the air-dried microspheres. This decreased density is indicative of the increased porosity of the microspheres. The swelling ratio of the lyophilized microspheres also increased considerably.

The following table shows a comparison of the average loading capacity of lyophilized composite microspheres (pre-frozen at −20° C. and −80° C.) as compared to air-dried, un-lyophilized composite microspheres using a protein model. The freeze-dried microspheres had considerably higher loading capacities. Alkaline phosphatase (ALP) loading was carried out at room temperature in small glass vials. The preweighed microspheres were incubated in 3 mL of a 1 mg/mL solution of ALP for 24 hours. Samples were taken after loading and the difference between the original concentration and the post-loading concentration were determined. The amount of ALP loaded was quantified using a standard assay based on the conversion of p-Nitrophenyl phosphate to p-Nitrophenyl by the ALP enzyme.

Average Loading Capacity of Composite Microspheres

| Sample | Loading (ug ALP/mg chitosan) |
|---|---|
| Freeze Dried @ −20 - Day 1 | 24.49 ± 5.16 |
| Air Dried - Day 1 | 5.43 |
| Freeze Dried @ −80 - Day 29 | 20.65 ± 3.2 |
| Air Dried - Day 29 | 3.16 ± 1.58 |
| Freeze Dried @ −80 - Day 46 | 20.50 ± 2.62 |
| Freeze Dried @ −20 - Day 46 | 24.56 ± 3.56 |
| Air Dried - Day 46 | 4.28 ± 2.52 |

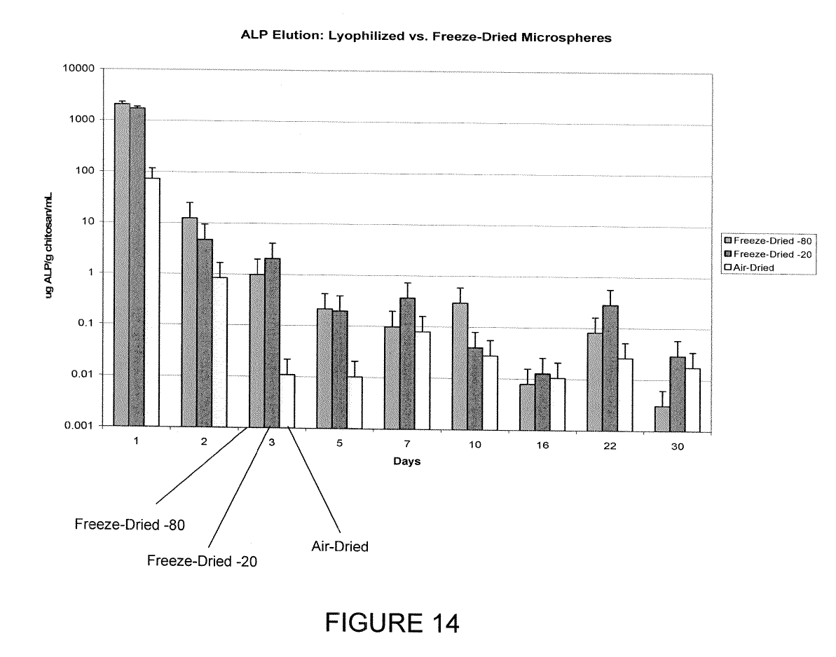

FIGS. 14-16 show how the release of therapeutic agents from composite microspheres can be extended by coating the microsphere. For this example, after the microspheres were loaded with ALP (as described above), they were placed in a 1× phosphate buffered saline (PBS) solution at room temperature. At certain time points, a sample of solution was taken. The remaining PBS solution was removed and fresh PBS solution added. The amount of ALP eluted was measured using the standard assay for the conversion of p-Nitrophenyl phosphate to p-Nitrophenyl by the ALP enzyme. The amount of ALP eluted was expressed in terms of μg ALP/mL/g chitosan.

FIG. 14 shows the elution over 30 days for lyophilized composite microspheres (pre-frozen at −20° C. and −80° C.) as compared to air-dried, un-lyophilized composite microspheres. The lyophilized composite microspheres released considerably more ALP over time than the air-dried microspheres.

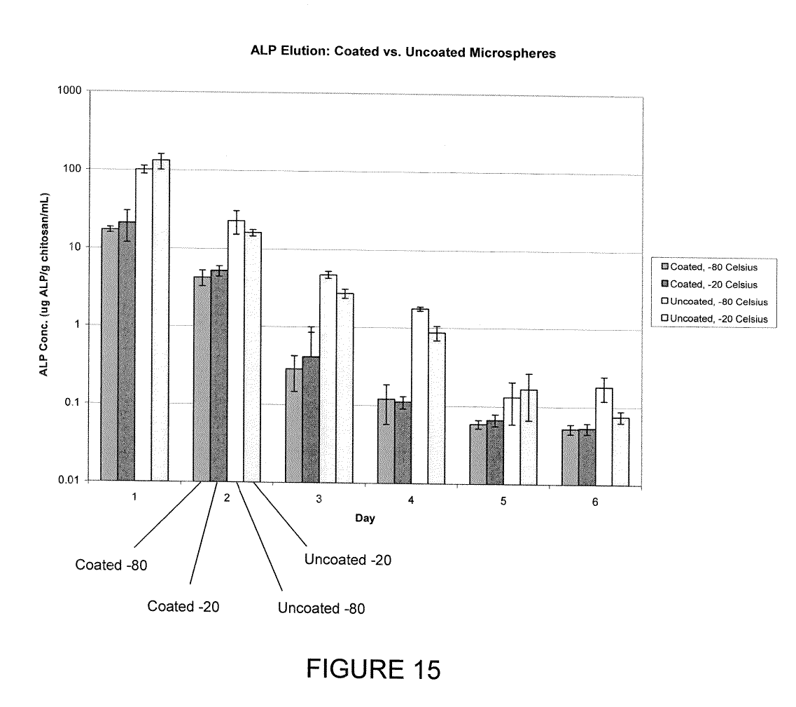

FIG. 15 shows the elution over 6 days of uncoated lyophilized composite microspheres (pre-frozen at −20° C. and −80° C.) as compared to the same lyophilized composite microspheres coated with an additional thin layer of chitosan after being loaded with ALP. In this example, a 2 wt % chitosan (92.3% DDA) solution was made and a thin layer added to the microspheres, although other weight concentration solutions may be used, as well as chitosan with a different DDA. The coating was allowed to dry overnight.

As seen in FIG. 15, the uncoated microspheres released more ALP during the six-day time period than the coated microspheres. As both the coated and uncoated microspheres are presumed to contain the same amount of ALP, this indicates that the coated microspheres at the end of the six-day time period will contain more ALP, thus indicating a more extended release profile.

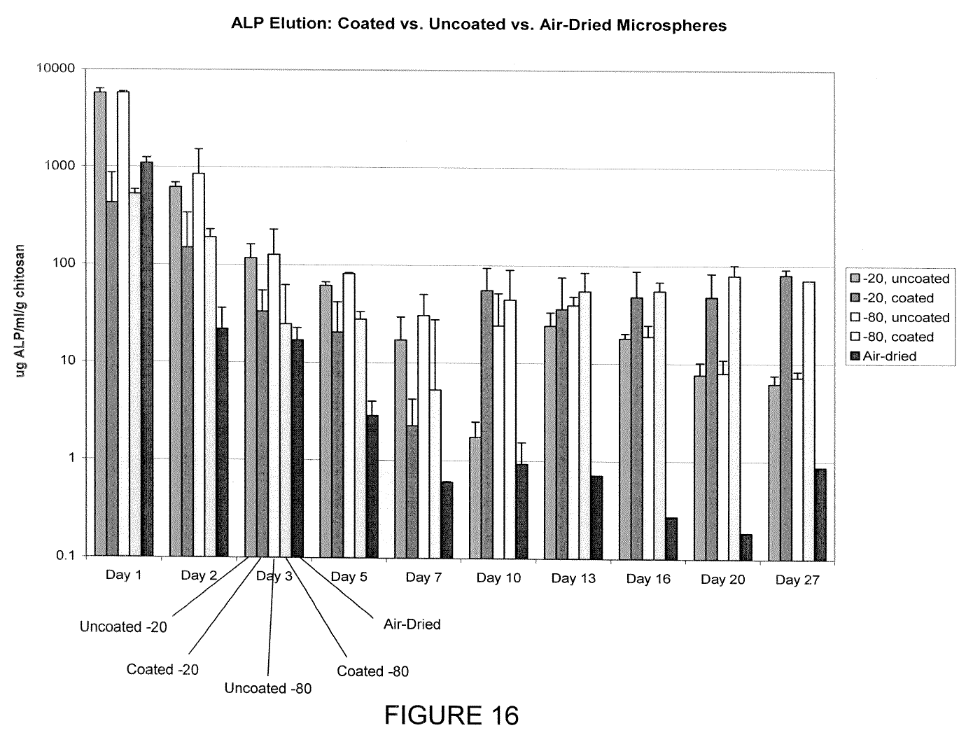

FIG. 16 shows that extended release profile. While the uncoated microspheres release more ALP initially, after approximately 7 to 10 days the coated microspheres release more ALP, and continue to maintain a fairly constant rate of release through the 27^{th} day.

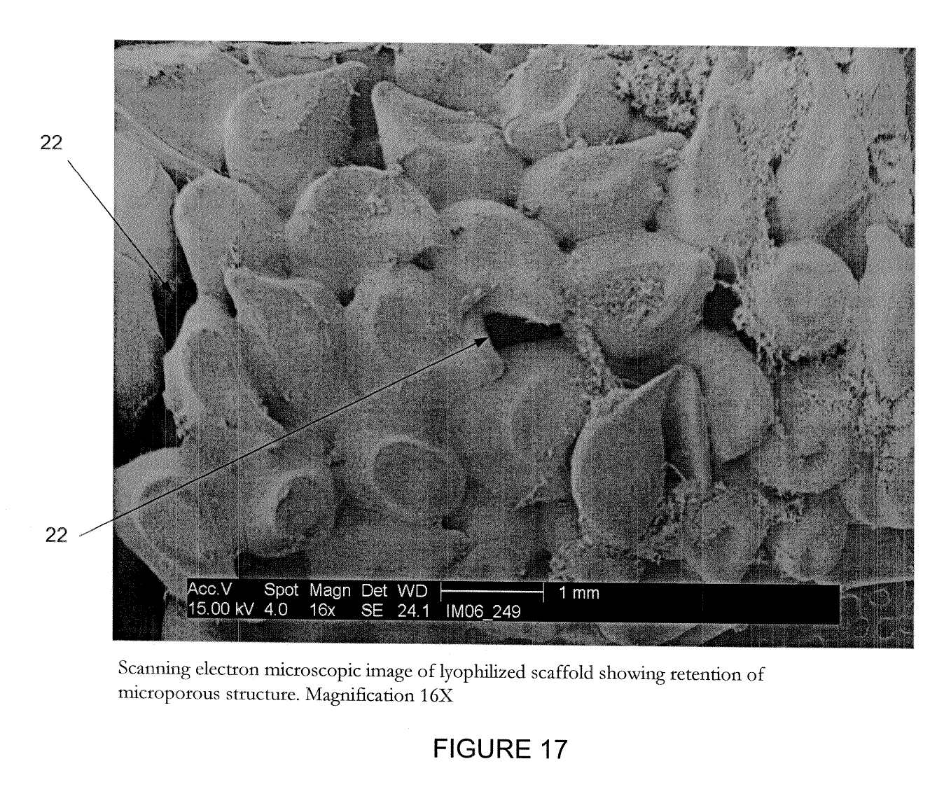

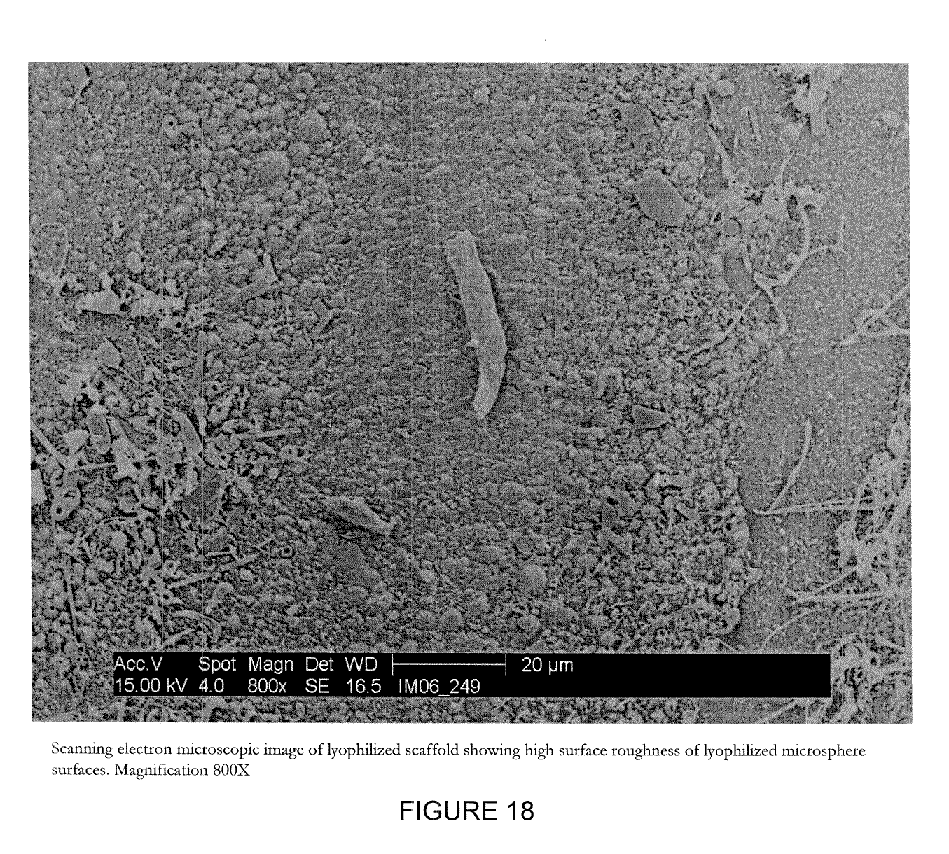

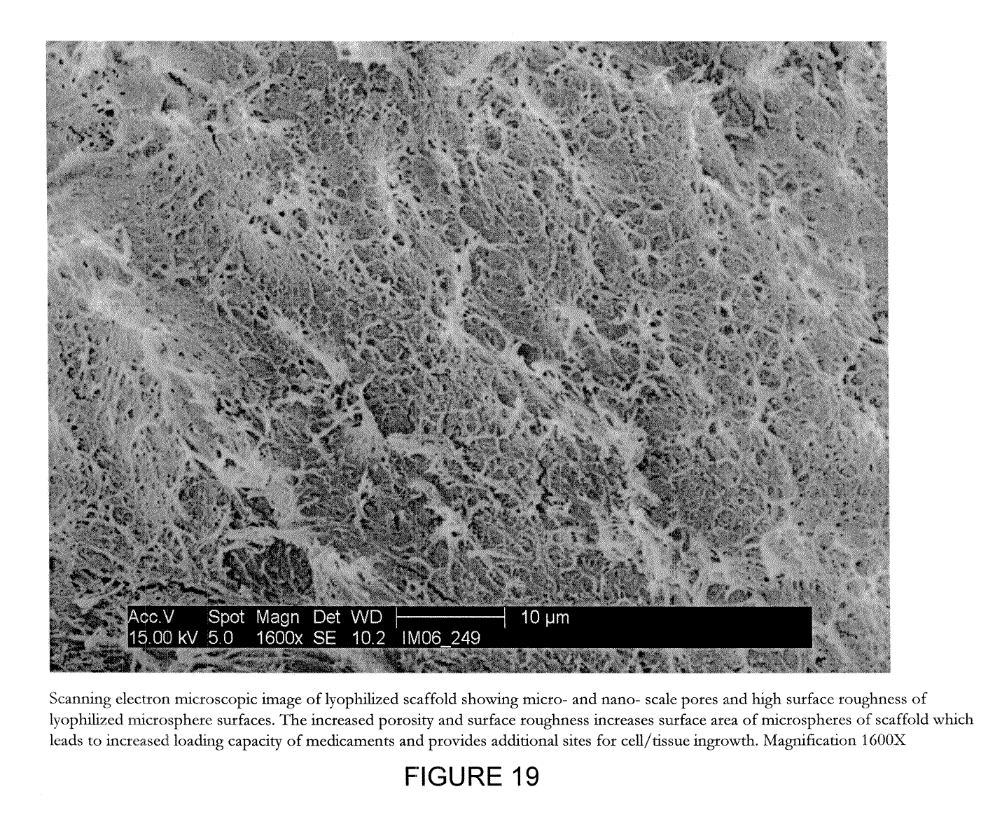

FIGS. 17-19 show SEM images of an exemplary embodiment of a lyophilized composite scaffold in accordance with one embodiment of the present invention. These images show the increased surface texture and porosity of the scaffold, which can be loaded with more medicaments due to the increased surface area. Microscale pores (or micropores) 22 can be seen that promote cell and tissue ingrowth.

Thus, it should be understood that the embodiments and examples have been chosen and described in order to best illustrate the principles of the invention and its practical applications to thereby enable one of ordinary skill in the art to best utilize the invention in various embodiments and with various modifications as are suited for the particular uses contemplated. Even though specific embodiments of this invention have been described, they are not to be taken as exhaustive. There are several variations that will be apparent to those skilled in the art. Accordingly, it is intended that the scope of the invention be defined by the claims appended hereto.

I claim:

1. A composite material for bone regeneration, comprising:
a plurality of microspheres comprising a composite of chitosan and crystalline calcium phosphate.

2. The composite material of claim 1, wherein the calcium phosphate crystals are approximately nano-sized.

3. The composite material of claim 1, wherein the microspheres are approximately 500 to 900 μm in diameter.

4. The composite material of claim 1, wherein the surface of the microspheres is roughened.

5. The composite material of claim 1, further wherein the microspheres are fused to form a scaffold.

6. The composite material of claim 5, wherein the scaffold has an average pore size of greater than approximately 100 μm.

7. The composite material of claim 5, wherein the scaffold has pore sizes between approximately 100 μm and 800 μm.

8. The composite material of claim 5, wherein the scaffold has a porosity of approximately 30 to 40%.

9. The composite material of claim 5, wherein the scaffold has a porosity of approximately 33 to 35%.

10. The composite material of claim 5, wherein the scaffold when hydrated has a compressive modulus of 9 MPa or greater.

11. The composite material of claim 1, wherein the microspheres are formed by co-precipitation.

12. The composite material of claim 1, where the composite microspheres contain one or more therapeutic agents.

13. The composite material of claim 12, wherein the composite microspheres are coated to extend the release of the therapeutic agents from the microspheres.

14. A method of forming a composite material for bone regeneration, comprising the steps of:
dripping a solution containing chitosan and CaCl₂ and NaH₂PO₄ into a NaOH/methanol solution to form chitosan-calcium phosphate microspheres;
removing the microspheres from the NaOH/methanol solution;
partially dissolving the surface of the microspheres; and
fusing the microspheres together to form a scaffold.

15. The method of claim 11, further comprising the step of constantly stirring the NaOH/methanol solution while the chitosan-solution is dripped therein.

16. The method of claim 11, further wherein the microspheres are left in the NaOH/methanol solution for approximately 24 hours prior to removal.

17. The method of claim 11, further wherein the step of partially dissolving the surface of the microspheres comprises washing the microspheres quickly with dilute acetic acid or other organic acid.

18. The method of claim 11, further comprising the step of
lyophilizing the scaffold.

19. The method of claim 18, further comprising the step of
loading medicament s or therapeutic agents into the scaffold.

20. The method of claim 19, further comprising the step of
coating the loaded scaffold.
